(19) United States
(12) Patent Application Publication
(10) Pub. No.: US 2009/0062549 A1
(43) Pub. Date: Mar. 5, 2009

(54) PROCESS FOR THE PREPARATION OF PRAMIPEXOLE AND NEW ANHYDROUS FORMS OF ITS DIHYDROCHLORIDE

(76) Inventors: Seema Kanwar, Chandigarh (IN); Killol Patel, Gujarat (IN); Keshav Deo, Haryana (IN); Mohan Prasad, Haryana (IN)

Correspondence Address: RANBAXY INC., 600 COLLEGE ROAD EAST, SUITE 2100, PRINCETON, NJ, 08540, US

(21) Appl. No.: 11/913,351

(22) PCT Filed: Apr. 26, 2006
(86) PCT No.: PCT/IB2006/001029

§ 371 Date: Jun. 24, 2008

(30) Foreign Application Priority Data
May 3, 2005 (IN) 1107/DEL/2005

(51) Int. Cl. C07D 277/82

(52) U.S. Cl. 548/164

(57) ABSTRACT

The present invention provides processes for the preparation of (−) pramipexole or an acid addition salt thereof of Formula I. The present invention further provides for the novel polymorphic Forms A and B of anhydrous pramipexole dihydrochloride.

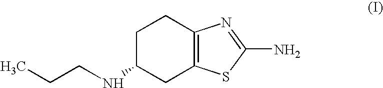

### FIELD OF THE INVENTION

The present invention provides processes for the preparation of (−) pramipexole or an acid addition salt thereof of Formula I.

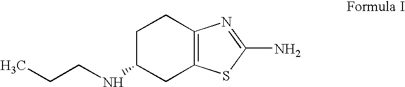

The present invention further provides for the novel polymorphic Forms A and B of anhydrous pramipexole dihydrochloride.

### BACKGROUND OF THE INVENTION

Pramipexole (“Formula I”) or chemically (S)-2-amino-4,5,6,7-tetra-hydro-6-(propylamino)benzothiazole, is indicated for the treatment of the symptoms of idiopathic Parkinson's disease. Pramipexole is commercially available in the dihydrochloride salt form as a monohydrate.

U.S. Pat. No. 4,886,812 discloses pramipexole or an acid addition salt thereof. The '812 patent further provides various processes for the preparation of pramipexole.

The Journal of Medical Chemistry 30, 494-498, (1987), describes a process for the preparation of optically pure pramipexole. It also discloses a process for the resolution of racemic 2,6-diamino intermediate using L-(+)-tartaric acid as a chiral auxiliary. The so obtained single enantiomer precursor is further converted to pramipexole by a two-step reaction, which involves N-acylation and reduction.

WO 02/022590 discloses a process for the preparation of pramipexole that involves reacting 6-substituted 2-amino-4,5,6,7-tetrahydrobenzothiazole with an amine in the presence of a reducing agent. The 6-substituted group may be an alkoxy, alkylenedioxy or an oxo group.

WO 02/022591 discloses a process for the resolution of pramipexole that involves reacting racemic pramipexole with an acid to form a monobasic acid addition salt which is further converted into the dibasic acid addition salt.

### SUMMARY OF THE INVENTION

In one general aspect there is provided a process for the preparation of (−) pramipexole or an acid addition salt thereof of Formula I. The process includes the steps of:
a) reacting the compound of Formula II

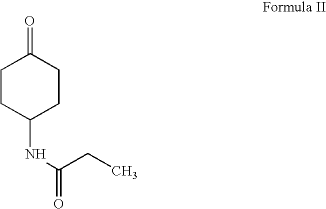

with bromine and thiourea in the presence of one or more organic solvents to obtain the compound of formula III or an acid addition salt thereof;

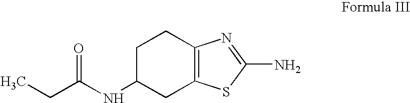

b) reducing the compound of Formula III with one or more reducing agents to obtain a racemic mixture of the compound of Formula I or an acid addition salt thereof; and
c) resolving the compound of Formula I with one or more chiral auxiliaries to get a (−) isomer of the compound of Formula I or an acid a salt thereof.

Embodiments of the process may include one or more of the following features. For example, the one or more reducing agents may be a borane-tetrahydrofuran complex or the organic solvent may include one or more of acetic acid, chloroform, carbon tetrachloride, tetrahydrofuran, diethyl ether or N,N-dimethylformamide. The process may further include using the (−) pramipexole or an acid addition salt thereof of Formula I in forming a pharmaceutical composition.

In another general aspect there is provided an anhydrous pramipexole dihydrochloride having moisture content of about 1% or less when measured by the Karl Fischer method.

Embodiments may include one or more of the following features or those described herein. For example, the anhydrous pramipexole dihydrochloride may be incorporated into a pharmaceutical composition

In another general aspect, there is provided a polymorphic Form A of anhydrous pramipexole dihydrochloride. Embodiments may include one or more of the following features or those described herein. For example, the polymorphic Form A may include an XRD pattern having characteristic 2theta values obtained at 6.3, 6.5, 24.7, 25.6, 26.8, and 27.7.

Embodiments of the polymorphic Form A may include one or more of the following features. For example, the polymorphic Form A of anhydrous pramipexole dihydrochloride may include 2theta values at 6.80, 12.04, 12.72, 12.96, 13.56, 13.98, 14.52, 14.86, 15.48, 15.80, 17.10, 17.82, 18.14, 18.36, 19.22, 19.54, 19.84, 19.96, 20.46, 20.72, 21.72, 22.38, 22.90, 23.10, 23.52, 24.04, 24.32, 25.02, 26.20, 26.46, 27.14, 28.36, 29.02, 29.38, 29.58, 29.88, 30.74, 31.32, 31.84, 32.22, 32.84, 33.66, 34.38, 34.92, 35.12, 35.56, 35.96, 36.44, 37.32, 37.60, 38.30, 38.72.

In another general aspect there is provided a polymorphic Form B of anhydrous pramipexole dihydrochloride. Embodiments may include one or more of the following features or those described herein. For example, the polymorphic Form B of anhydrous pramipexole dihydrochloride may include an XRD pattern having characteristic 2theta values obtained at 6.4, 6.6, 26.5, and 28.4.

Embodiments of the polymorphic Form B may include one or more of the following features. For example, the polymorphic Form B of anhydrous pramipexole dihydrochloride may include 2theta values at 6.02, 12.10, 12.78, 13.64, 14.08, 14.56, 14.92, 15.54, 17.16, 17.88, 18.20, 18.42, 19.24, 19.58, 20.00, 20.54, 20.80, 21.78, 23.08, 23.46, 23.60, 24.08, 24.84, 25.74, 26.18, 26.86, 27.22, 27.54, 27.86, 29.10, 29.42, 30.00, 30.82, 31.40, 31.98, 32.30, 32.86, 33.74, 34.58, 35.04, 35.24, 35.62, 36.04, 37.40, 37.64, 38.78.

In another general aspect there is provided a pharmaceutical composition that includes anhydrous pramipexole dihydrochloride and one or more pharmaceutically acceptable excipients.

Embodiments of the pharmaceutical composition may include one or more of the following features. For example, the anhydrous pramipexole dihydrochloride may be polymorphic Form A or Form B of anhydrous pramipexole dihydrochloride. The pharmaceutical composition may include a mixture of polymorphic Form A and Form B of anhydrous pramipexole dihydrochloride.

In another general aspect there is provided a method of treating the symptoms of idiopathic Parkinson's disease. The method includes administering to a mammal in need thereof a therapeutically effective amount of anhydrous pramipexole dihydrochloride.

Embodiments of the method may include one or more of the following features. For example, the anhydrous pramipexole dihydrochloride may be Form A or Form B anhydrous pramipexole dihydrochloride. The anhydrous pramipexole dihydrochloride administered may be a mixture of polymorphic Form A and Form B of anhydrous pramipexole dihydrochloride.

In yet another general aspect there is provided a method of treating the symptoms of idiopathic Parkinson's disease. The method includes administering to a mammal in need thereof a therapeutically effective amount of anhydrous pramipexole dihydrochloride having moisture content of about 1% or less when measured by the Karl Fischer method.

Embodiments of the method may include one or more of the following features. For example, the anhydrous pramipexole dihydrochloride may be Form A or Form B anhydrous pramipexole dihydrochloride. The anhydrous pramipexole dihydrochloride may be a mixture of polymorphic Form A and Form B of anhydrous pramipexole dihydrochloride.

The details of one or more embodiments of the inventions are set forth in the description below. Other features, objects and advantages of the inventions will be apparent from the description and claims.

### BRIEF DESCRIPTION OF THE DRAWINGS

FIG. 1 depicts the X-Ray Diffraction Pattern (XRD) of polymorphic Form A of anhydrous pramipexole dihydrochloride.

FIG. 2 depicts Differential Scanning Calorimetric (DSC) thermogram of polymorphic Form A of anhydrous pramipexole dihydrochloride.

FIG. 3 depicts the X-Ray Diffraction Pattern (XRD) of polymorphic Form B of anhydrous pramipexole dihydrochloride.

FIG. 4 depicts Differential Scanning Calorimetric (DSC) thermogram of polymorphic Form B of anhydrous pramipexole dihydrochloride.

### DETAILED DESCRIPTION OF THE INVENTION

The present inventors have now found a direct and cost-effective process for the preparation of (−) pramipexole or an acid addition salt thereof of Formula I,

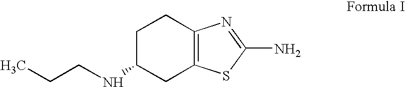

wherein the process does not require the introduction and/or removal of a protective group. Instead the propionyl group serves as a protective group and a direct precursor for the desired propyl group.

The present inventors have further found that pramipexole dihydrochloride exists in an anhydrous form having a moisture content of about 1% w/w or less when determined by Karl Fischer analysis. Two characteristic forms of anhydrous pramipexole dihydrochloride have been isolated. These two forms are designated as Form A and Form B and remain stable under normal storage conditions.

The term “compound of Formula I or acid addition salt thereof” in the present invention refers to a compound of Formula I, or a monobasic or dibasic acid addition salt thereof. The acid addition salt may include inorganic or organic acid addition salts. The dibasic acid addition salt may be a mixed acid addition salt of two different acids. The term may also include hydrates, solvates and enantiomers of the compound of Formula I or acid addition salt thereof.

The first aspect of the invention provides a process for the preparation of (−) pramipexole or an acid addition salt thereof of Formula I. The process includes:
a) reacting the compound of Formula II

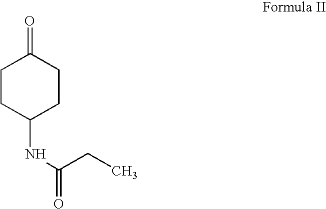

with bromine and thiourea to obtain the compound of formula III or an acid addition salt thereof;

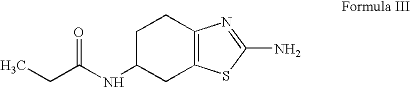

b) reducing the compound of Formula III with a reducing agent to obtain a racemic mixture of the compound of Formula I or an acid addition salt thereof; and
c) resolving the compound of Formula I, with a chiral auxiliary to obtain the (−) isomer of the compound of Formula I or an acid addition salt thereof.

In a second aspect of the invention there is provided a process for the preparation of racemic pramipexole or an acid addition salt thereof. The process includes reducing the compound of Formula III;

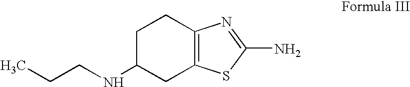

wherein the reducing agent used is borane-tetrahydrofuran complex.

Bromine is added drop wise to a solution of a compound of Formula II in an organic solvent for about 2-5 hours and the mixture is stirred at an ambient temperature. Suitable organic solvents include acetic acid, chloroform, carbon tetrachloride, tetrahydrofuran, diethyl ether or N,N-dimethylformamide. Thiourea is added to the reaction mixture and the resulting mixture is refluxed for about 1 to about 4 hours. The mixture is cooled and the solid obtained is filtered and washed to provide a compound of Formula III or an acid addition salt thereof.

The compound of Formula III is put into an organic solvent and a reducing agent is added drop wise under nitrogen at about room temperature. The resulting mixture is stirred at from about 35° C. to about 60° C., cooled and acidified.

Suitable solvents may include one or more of tetrahydrofuran, diethyl ether, diglyme or an alkanol. After the completion of reduction, water followed by concentrated hydrochloric acid, is added to the reaction mass and the solvent is evaporated. The aqueous residue is basified and extracted with ethyl acetate. The solid obtained is filtered, washed and air-dried to provide the compound of Formula I.

The ‘reducing agent’ may include one or more of borane, borane-tetrahydrofuran, borane-dimethyl sulfide, sodium borohydride and Boron trifluoride-etherate complex and mixtures thereof. The racemic compound of Formula I is resolved using a chiral auxiliary to obtain (−) isomer of Formula I or acid addition salt thereof.

A third aspect of the present invention provides anhydrous pramipexole dihydrochloride characterized by having moisture content of about 1% or less when measured by Karl Fischer method.

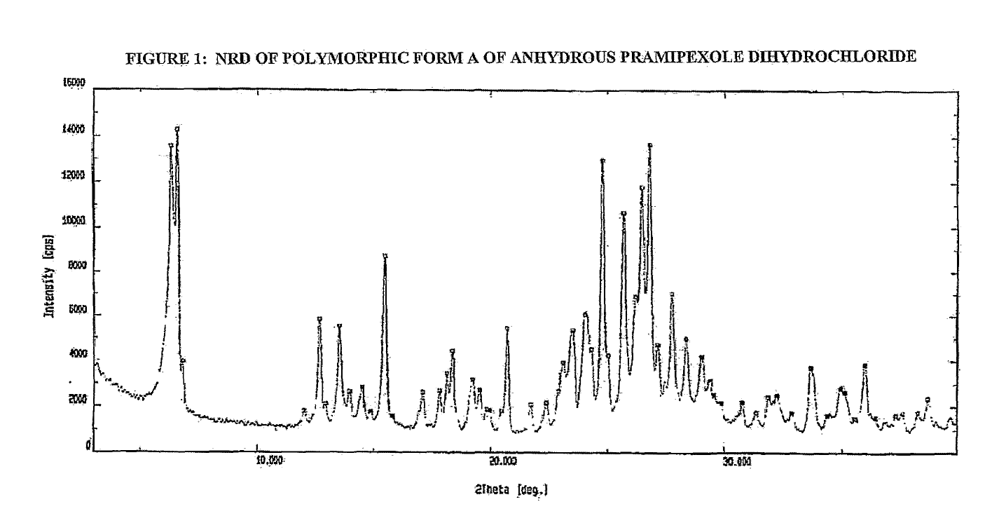

A fourth aspect of the present invention provides for polymorphic Form A of anhydrous pramipexole dihydrochloride having X-Ray Powder Diffraction (XRD) pattern as depicted in FIG. 1 of the accompanied drawings. The characteristic 2theta values are observed at 6.02, 6.40, 6.66, 12.10, 12.78, 13.64, 14.08, 14.56, 14.92, 15.54, 17.16, 17.88, 18.20, 18.42, 19.24, 19.58, 20.00, 20.54, 20.80, 21.78, 23.08, 23.46, 23.60, 24.08, 24.84, 25.74, 26.18, 26.54, 26.86, 27.22, 27.54, 27.86, 28.44, 29.10, 29.42, 30.00, 30.82, 31.40, 31.98, 32.30, 32.86, 33.74, 34.58, 35.04, 35.24, 35.62, 36.04, 37.40, 37.64, 38.78.

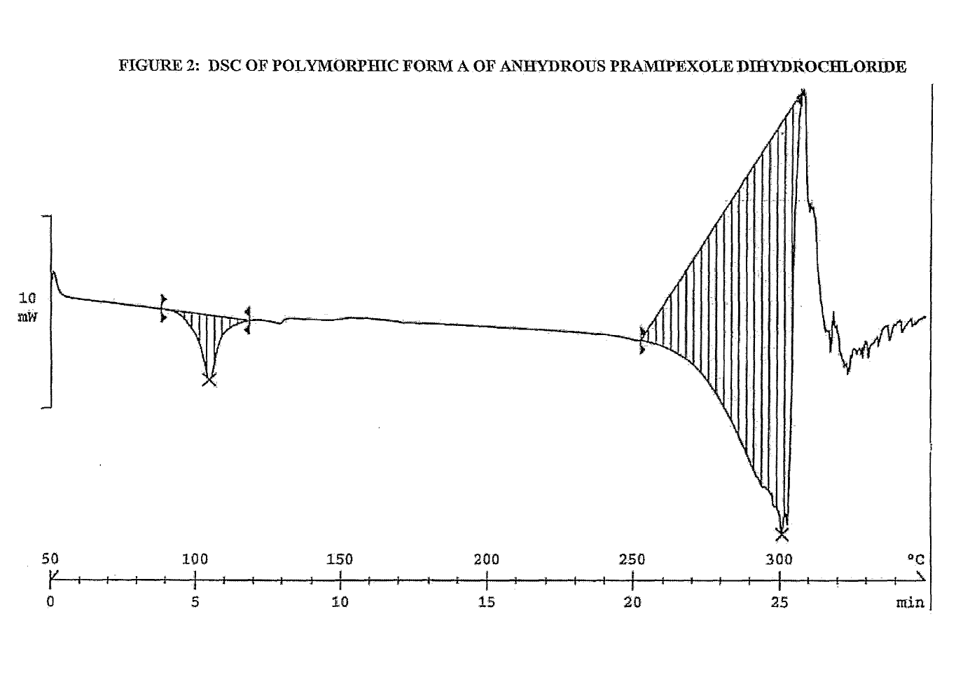

The polymorphic Form A of anhydrous pramipexole dihydrochloride has a characteristic Differential Scanning Calorimetric (DSC) thermogram as depicted in FIG. 2 of the accompanied drawings. The characteristic endothermic absorptions are observed between about 220° C. to about 310° C. and between about 315° C. and about 350° C. Form A of anhydrous pramipexole dihydrochloride has a moisture content of about 1% or less.

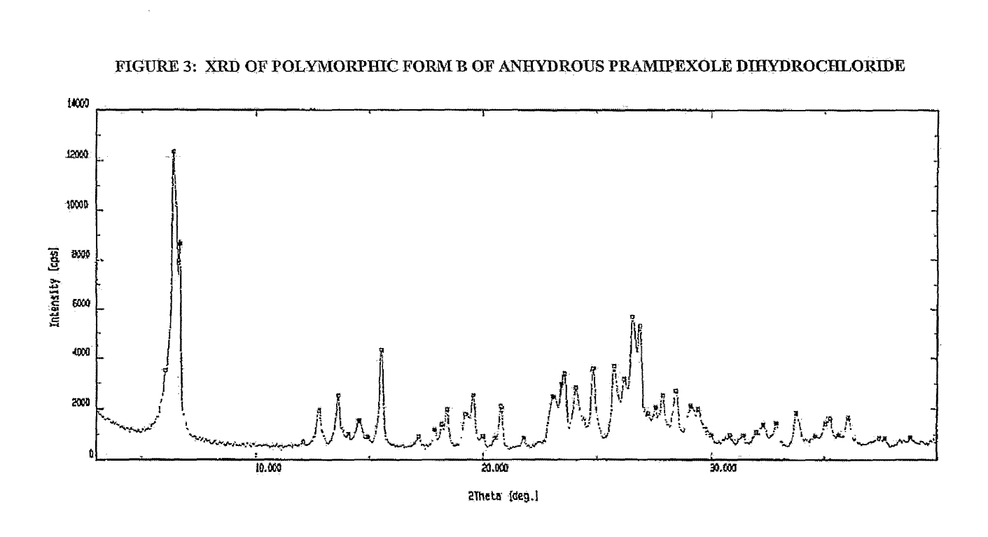

A fifth aspect of the present invention provides for polymorphic Form B of anhydrous pramipexole dihydrochloride having an X-Ray Powder Diffraction (XRD) pattern as depicted in FIG. 3. The polymorphic Form B includes characteristic 2theta values that are obtained at 6.32, 6.58, 6.80, 12.04, 12.72, 12.96, 13.56, 13.98, 14.52, 14.86, 15.48, 15.80, 17.10, 17.82, 18.14, 18.36, 19.22, 19.54, 19.84, 19.96, 20.46, 20.72, 21.72, 22.38, 22.90, 23.10, 23.52, 24.04, 24.32, 24.78, 25.02, 25.68, 26.20, 26.46, 26.80, 27.14, 27.76, 28.36, 29.02, 29.38, 29.58, 29.88, 30.74, 31.32, 31.84, 32.22, 32.84, 33.66, 34.38, 34.92, 35.12, 35.56, 35.96, 36.44, 37.32, 37.60, 38.30, 38.72.

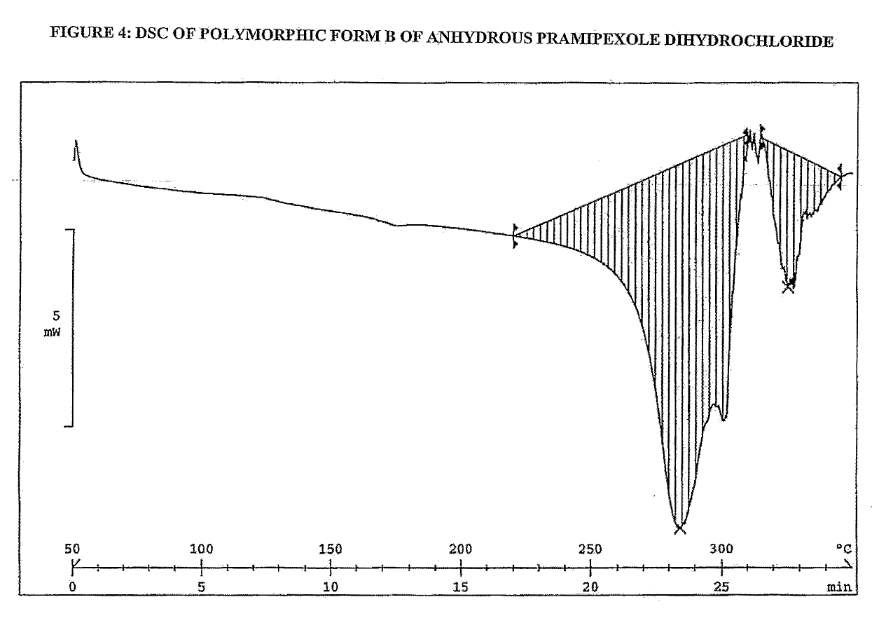

The polymorphic Form B of anhydrous pramipexole dihydrochloride has a characteristic Differential Scanning Calorimetric (DSC) thermogram as depicted in FIG. 4 wherein the characteristic endothermic absorptions are observed between about 90° C. to about 120° C. and between about 255° C. to about 310° C.

A sixth aspect of the present invention provides pramipexole dibasic acid addition salt having less than detectable quantity of the monobasic acid addition salt. In order to incorporate pramipexole or salt thereof in the finished dosage form it is very important to know the exact content of the pramipexole base in the bulk active. Pramipexole has a tendency to form monobasic, as well as dibasic acid addition salts with organic or inorganic acids. The approved drug in the market is pramipexole dihydrochloride which is a dibasic acid addition salt with hydrochloric acid. A contamination with monobasic acid addition salt in the dibasic acid addition salt would complicate the issue for a formulation chemist as he would not be exactly certain on the required weight of the required bulk active in order to prepare the finished dosage form.

The examples are provided to illustrate particular aspects of the disclosure and are not intended to limit the scope of the present invention as defined by the claims.

### EXAMPLE 1

### Preparation of 4-Propionamide Cyclohexanol

To a solution of 4-amino cyclohexanol (100 g) in dichloromethane (2000 ml) kept at 5-10° C. propionic anhydride (114 g) was added drop wise for 1 hour. The temperature was raised to about 20° C. to about 25° C. and the reaction mixture was stirred at this temperature for about 5-6 hours. After completion of the reaction, the mixture was cooled to about 5° C. to about 10° C. and filtered to obtain the title compound.

### EXAMPLE 2

### Preparation of 4-Propionamido Cyclohexanone

To a solution of 4-propionamido-cyclohexanol (100 g) in acetone (1000 ml), Jones

Reagent (prepared from chromic acid (66.54 g), sulphuric acid (96.28 g) and water (400 ml)) were added while at a temperature of between about 5° C. to about 10° C. The mixture was stirred for 5-6 hours. After completion of the reaction, excess reagent was quenched by the addition of isopropanol (400 ml). The residue of Jones reagent was filtered and the solid was washed with ethyl acetate (100 ml×3). The solvent was removed under reduced pressure and the residue was again extracted with ethyl acetate (200 ml×3). The contents were stirred for 30 minutes at a temperature of between about 45° C. to about 50° C. and the upper portion was decanted and concentrated completely under vacuum to obtain a solid. To the residue ethyl acetate (100 ml) was charged and stirred at a temperature of between about 45° C. to about 50° C. for 30 minutes to get a clear solution and then hexane (200 ml) was charged. The mixture was cooled to a temperature of between about 0° C. to about 5° C. in 2 hours and filtered at between about 0° C. to about 5° C. to get the title compound.

### EXAMPLE 3

### Preparation of 2-amino-6-propionylamino-4,5,6,7-tetrahydro Benzothiazole

Bromine (160 g) was added drop wise to a solution of 4-propionamido-cyclohexanone (155 g) in glacial acetic acid (1500 ml) in 3 hours and the mixture was stirred for 1-2 hours at ambient temperature. Thiourea (152.0 g) was added to the reaction mixture and the resulting mixture was refluxed for 2-3 hours. After cooling, the crystals of 2-amino-6-(N-propionamido)-4,5,6,7-tetrahydro-benzothiazole-hydrobromide precipitated were filtered and washed with ethyl acetate.

2-amino-6-(N-propionamido)-4,5,6,7-tetrahydro-benzothiazole-hydrobromide was charged to water (620 ml) to get a clear solution. To this a 50% sodium hydroxide solution (39 g in 110 ml water) was added drop wise at a temperature of between about 5° C. to about 10° C. The solid precipitated was filtered and washed with isopropanol (155 ml) to get the title compound.

### EXAMPLE 4

### Preparation of (±)-2-Amino-6-(N-propylamino)-4,5,6,7-tetrahydro Benzothiazole

To a solution of 2-amino-6-(N-propionamido)-4,5,6,7-tetrahydro-benzothiazole (2.25 g) in anhydrous tetrahydrofuran (25 ml), borane-tetrahydrofuran complex (50 ml, 1M solution in tetrahydrofuran) was added drop wise under nitrogen at room temperature. The resulting mixture was stirred at a temperature of about 50° C. for 1 hour, cooled, and then water (5 ml) and concentrated aqueous hydrochloric acid (10 ml) were added. The tetrahydrofuran was evaporated and 25% aqueous sodium hydroxide (30 ml) was added to the water phase. The desired compound was extracted from the reaction mixture by using ethyl acetate (100 ml×3). The organic phase was dried and concentrated. Hexane (10 ml) was added to the resulting oily residue and stirred for 15 minutes. The solid obtained was filtered and air-dried to get the title compound.

Yield: 1.9 g

### EXAMPLE 5

### Preparation of (−)-2-Amino-6-(N-propylamino)-4,5,6,7-tetrahydro Benzothiazole-dihydrochloride

### (a) Preparation of (±)-2-amino-6-(N-propylamino-4,5,6,7-tetrahydrobenzo thiazole monohydrochloride

((±)-2-amino-6-(N-propylamino)-4,5,6,7-tetrahydrobenzothiazole (4.2 g) was dissolved in hot methanol (15 ml) and hydrochloric acid (1.65 ml) was added. The obtained suspension was stirred, cooled and the precipitated solid was filtered off. The cake was washed with cold methanol and air-dried to give the title compound.

Yield: 4.2 g

### (b) A mixed salt of pramipexole monohydrochloride with L-(+)-tartaric acid

(±)-2-amino-6-propylamino-4,5,6,7-tetrahydrobenzothiazole monohydrochloride was dissolved in hot methanol (23 ml) and L-(+)-tartaric acid (1.80 g) was added. White crystals precipitated, and the mixture was cooled and the crystals were removed by suction. The crystals were washed with cold methanol and dried to give a solid, which was recrystallized from hot methanol to give the title compound.

Yield: 2.2 g

### (c) Liberation of (S)-pramipexole Freebase

The pramipexole monohydrochloride monotartrate from the step (b) was dissolved in water (10 ml) and cooled to about 10° C. To this solution a solution of potassium hydroxide (5 g in 10 ml DI water) was added. The mixture was stirred at a temperature of about 10° C. for 30 minutes and the white crystals were filtered and washed with cold water. The cake was dried to give white crystals of the title compound.

Yield: 0.95 g

### (d) (S)-Pramipexole dihydrochloride

The crystals from the step (c) were dissolved in ethanol (7 ml) and ethanolic hydrochloride was added drop wise at a temperature of about 10° C. and the mixture was stirred at this temperature for about 1 hour. The crystals formed were filtered and washed with cold methanol. The cake was dried to give white crystals of the title compound.

Yield: 1.1 g

While the present invention has been described in terms of its specific embodiments, certain modifications and equivalents will be apparent to those skilled in the art and are included within the scope of the present invention.

We claim:

1. A process for the preparation of (−) pramipexole or an acid addition salt thereof of Formula I, wherein the process comprises the steps of:
a) reacting the compound of Formula II 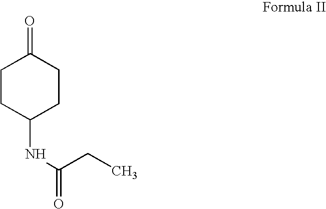
with bromine and thiourea in the presence of at least one organic solvents to obtain the compound of formula III or an acid addition salt thereof; 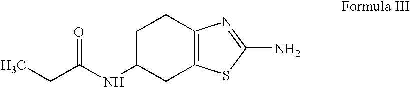
b) reducing the compound of Formula III with at least one reducing agents to obtain a racemic mixture of the compound of Formula I or an acid addition salt thereof; 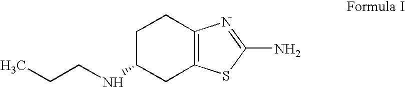
and
c) resolving the compound of Formula I with at least one chiral auxiliary to get the (−) isomer of the compound of Formula I or an acid a salt thereof.

2. The process according to claim 1, wherein borane-tetrahydrofuran complex is one of the reducing agents.

3. The process according to claim 1, wherein the organic solvent is selected from acetic acid, chloroform, carbon tetrachloride, tetrahydrofuran, diethyl ether, N,N-dimethylformamide or a mixture thereof.

4. The process according to claim 1, further comprising using (−) pramipexole of Formula I so prepared or an acid addition salt thereof in preparing a pharmaceutical composition.

5-20. (canceled)
